(19) United States
(12) Patent Application Publication
(10) Pub. No.: US 2020/0294633 A1
(43) Pub. Date: Sep. 17, 2020

(54) Facilitating Interoperability Across Health Information Systems

(71) Applicants: Vipin Namboodiri, Bangalore (IN); Boppana Visweswara Rao, Bangalore (IN)

(72) Inventors: Vipin Namboodiri, Bangalore (IN); Boppana Visweswara Rao, Bangalore (IN)

(73) Assignee: Ricoh Company, Ltd., Tokyo (JP)

(21) Appl. No.: 16/355,616

(22) Filed: Mar. 15, 2019

(51) Int. Cl. G16H 10/60; G06F 16/25; G06F 16/23; G06F 17/27

(52) U.S. Cl. CPC G16H 10/60; G06F 17/2705; G06F 16/2365; G06F 16/258

(57) ABSTRACT

A system and method that provides an interface to facilitate interoperability between health information systems is disclosed. The method includes receiving an electronic medical record in a first data format from a first health care server, parsing one or more elements of the electronic medical record in the first data format, mapping the one or more elements of the electronic medical record from the first data format to an intermediate data format using a schema mapping associated with the first data format and the intermediate data format, converting the electronic medical record from the intermediate data format to a second data format of a second health care server, and transmitting the converted electronic medical record in the second data format to the second health care server.

### BACKGROUND

### Field of the Invention

The specification generally relates to providing an interface for facilitating interoperability to communicate, exchange, and use electronic health information across health information systems. In particular, the specification relates to a system and method for facilitating interoperability by transforming and integrating electronic health information across different health information systems.

### Description of the Background Art

Typically, hospitals and other health care organizations build and maintain health information systems in isolation where many different applications run on a variety of operating systems and hardware platforms to manage electronic health information. Even though the applications in the health information systems may support network connectivity, the exchange of electronic health information may be done using proprietary protocols. This makes the integration of electronic health information, particularly from different vendors of the health information systems, very difficult. Moreover, the costs and risks associated with integrating the electronic health information distributed across the health information systems is increased.

Previous attempts at integrating electronic health information have deficiencies. For example, one method is to enable the health information systems to store the electronic health information in a standard format. Unfortunately, this approach is expensive and impractical because the business needs of different health information systems may vary.

### SUMMARY

The techniques introduced herein overcome the deficiencies and limitations of the prior art, at least in part, with a system and method for providing an interface to facilitate interoperability between health information systems. In one embodiment, the system includes one or more processors and a memory storing instructions which when executed cause the one or more processors to receive, from a first health care server, an electronic medical record in a first data format, parse one or more elements of the electronic medical record in the first data format, map the one or more elements of the electronic medical record from the first data format to an intermediate data format using a schema mapping associated with the first data format and the intermediate data format, convert the electronic medical record from the intermediate data format to a second data format of a second health care server, and transmit the converted electronic medical record in the second data format to the second health care server.

Other aspects include corresponding methods, systems, apparatuses, and computer program products for these and other innovative aspects.

The features and advantages described herein are not all-inclusive and many additional features and advantages will be apparent in view of the figures and description. Moreover, it should be noted that the language used in the specification has been principally selected for readability and instructional purposes and not to limit the scope of the techniques described.

### BRIEF DESCRIPTION OF THE DRAWINGS

The techniques introduced herein are illustrated by way of example, and not by way of limitation in the figures of the accompanying drawings in which like reference numerals are used to refer to similar elements.

FIG. 1 is a high-level block diagram illustrating one embodiment of a system for providing an interface to facilitate interoperability between health information systems.

FIG. 2A is a block diagram illustrating one embodiment of a computing device including an EMR interface application.

FIG. 2B is a block diagram illustrating one embodiment of the EMR interface application.

FIG. 3 is a block diagram illustrating another embodiment of the EMR interface application 103.

FIG. 4 is a flow diagram illustrating one embodiment of an example method for providing an interface to facilitate interoperability between health information systems.

FIG. 5 is a flow diagram illustrating one embodiment of an example method for converting a system of measurement of the elements of a data object.

FIG. 6 is a flow diagram illustrating one embodiment of an example method for converting a medical coding classification system of the elements of a data object.

### DETAILED DESCRIPTION

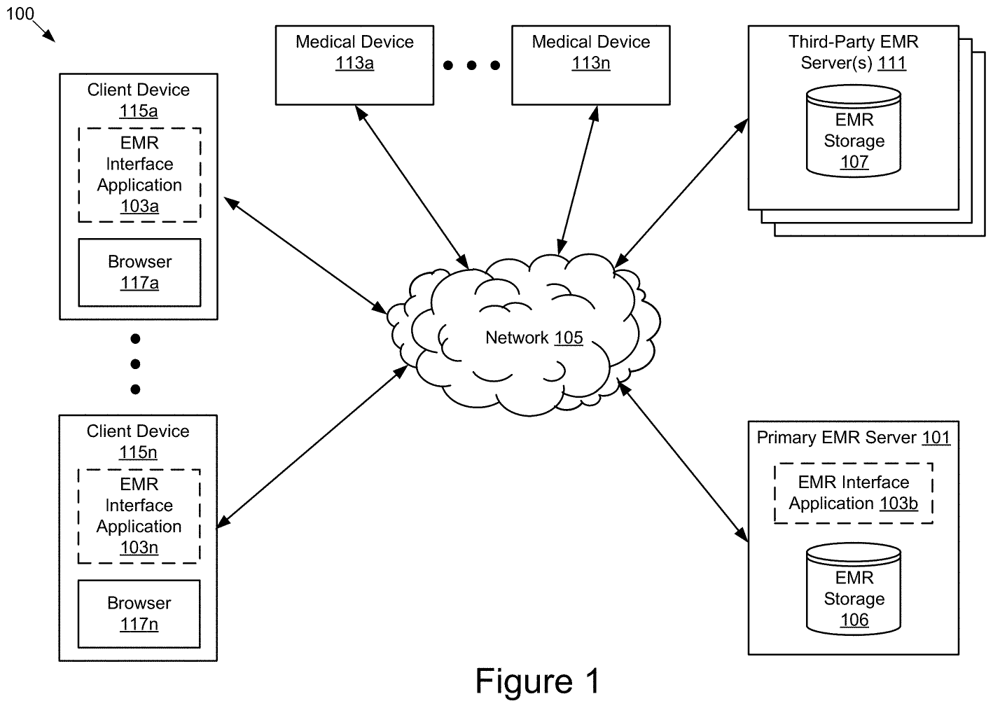

FIG. 1 is a high-level block diagram illustrating one embodiment of a system 100 for providing an interface to facilitate interoperability between health information systems. The illustrated system 100 may have one or more client devices 115a . . . 115n, which can be accessed by users, one or more medical devices 113a . . . 113n, a primary electronic medical record (EMR) server 101, and one or more third-party EMR servers 111. In FIG. 1 and the remaining figures, a letter after a reference number, e.g., “115a,” represents a reference to the element having that particular reference number. A reference number in the text without a following letter, e.g., “115,” represents a general reference to instances of the element bearing that reference number. In the illustrated embodiment, these entities of the system 100 are communicatively coupled via a network 105.

The network 105 can be a conventional type, wired or wireless, and may have numerous different configurations including a star configuration, token ring configuration, or other configurations. Furthermore, the network 105 may include a local area network (LAN), a wide area network (WAN) (e.g., the Internet), and/or other interconnected data paths across which multiple devices may communicate. In some embodiments, the network 105 may be a peer-to-peer network. The network 105 may also be coupled to or include portions of a telecommunications network for sending data in a variety of different communication protocols. In some embodiments, the network 105 may include Bluetooth communication networks or a cellular communications network for sending and receiving data including via short messaging service (SMS), multimedia messaging service (MMS), hypertext transfer protocol (HTTP), direct data connection, WAP, email, etc. Although FIG. 1 illustrates one network 105 coupled to the client devices 115, the medical devices 113, the primary EMR server 101, and third-party EMR servers 111, in practice one or more networks 105 can be connected to these entities.

In some embodiments, the primary EMR server 101 and each of the one or more third-party EMR servers 111 may be either a hardware server, a software server, or a combination of software and hardware. The primary EMR server 101 and each of the one or more third-party EMR servers 111 may be, or may be implemented by, a computing device including a processor, a memory, applications, a database, and network communication capabilities. In general, the EMR servers 101 and 111 may be health care servers administered by different health care organizations for collecting, storing, managing, and transmitting electronic health information of patients.

In the example of FIG. 1, the components of the primary EMR server 101 are configured to implement an EMR interface application 103b and an EMR storage 106 described in more detail below. In some embodiments, the primary EMR server 101 may be a cloud-based (e.g., web-based) health care server associated with servicing requests from a client device 115 via the browser 117. In some embodiments, the primary EMR server 101 may also include a dedicated EMR application or medical practice management software (PMS) application (not shown) for creating, processing, and storing an electronic medical record (EMR) including medical history, admissions, discharges, transfers, appointments, allergies, lab results, treatment plans, prescriptions, scheduling information, patient billing information, etc. of a patient in the EMR storage 106. An electronic medical record (EMR) may be defined as an electronic record of health-related information on an individual that can be created, gathered, managed, and consulted by authorized personnel within a health care organization. In some embodiments, the primary EMR server 101 may store EMR data in a file format, such as Java Script Object Notation (JSON) file format in the EMR storage 106.

In some embodiments, the plurality of third-party EMR servers 111 may belong to different health care organizations. Each of the third-party EMR servers 111 may implement a PMS application (not shown) for handling health care data including EMR data of patients in a health care organization. For example, the PMS application may be from a vendor, such as OpenEMR®, Epic® EMR, Centricity® EMR, etc. In some embodiments, the plurality of third-party EMR servers 111 may store their own EMR data in the associated EMR storage 107 in a file format that is different from the primary EMR server 101. For example, the EMR data may be stored using health level-7 (HL7) message protocol. HL7 refers to an American National Standard Institute (ANSI) accredited standards organization that publishes data specifications for the electronic exchange of health care-related data. The HL7 specification documents provide a framework for communicating, exchanging, integrating, sharing and/or retrieval of EMR data. There are several version of HL7, such as versions 2.x and version 3.

In some embodiments, the servers 101 and 111 send and receive data to and from other entities of the system 100 via the network 105. For example, the primary EMR server 101 sends and receives data including EMR to and from the client device 115 and the third-party servers 111. When the client device 115 transmits information about a patient, the primary EMR sever 101 may update the electronic medical record of the patient in the EMR storage 106. Although only a single primary EMR server 101 is shown in FIG. 1, it should be understood that there may be any number of primary EMR servers 101 or a server cluster.

The client device 115 may be a computing device that includes a memory, a processor, for example a laptop computer, a desktop computer, a tablet computer, a mobile telephone, a smartphone, a personal digital assistant (PDA), a mobile email device, a webcam, a user wearable computing device, a television, or any other electronic device capable of accessing a network 105. The client device 115 provides general graphics and multimedia processing for any type of application. The client device 115 includes a display for viewing information provided by the servers 101 and 111. The client device 115 includes a browser 117. The browser 117 is code and routines stored in a non-transitory computer-readable memory of the client device 115 and is executed by a processor of the client device 115 for accessing the functionality and data provided by the servers 101 and 111 via the network 105.

While FIG. 1 illustrates two client devices 115a and 115n, the disclosure applies to a system architecture having one or more client devices 115. The client device 115 is adapted to send and receive data to and from the servers 101 and 111. For example, the client device 115 may be accessed by a patient for querying the server 101 about the patient information stored in the EMR storage 106. In another example, the client device 115 may be accessed by an authorized personnel (e.g., a registered nurse, a lab technician, a receptionist, etc.) to register a patient's information with the servers 101 and 111.

The medical device 113 may include, but not limited to, a stethoscope, a blood pressure meter, a pulse oximeter, a thermometer, an ophthalmoscope, a weight and height scale, an otoscope, a camera, a telecardiology device (e.g. an ECG machine), a telepathology device (e.g. a microscope), a teledermatology device (e.g. a high-resolution camera), a teleradiology device (e.g. an ultrasound machine), etc. associated with one or more health care organizations. An authorized personnel who is trained to use the medical device 113 may obtain the patient's medical information. In some embodiments, the medical device 113 may work with the client device 115 to allow authorized personnel to communicate with other entities of the system 100. For example, the client device 115 receives a report associated with a patient including a medical test result from the medical device 113, and sends the report to the primary EMR server 101 for updating the EMR of the patient in the EMR storage 106.

The EMR interface application 103 may include software and/or logic to provide the functionality for performing data transformation, coding system conversion, unit measurement conversion, and/or integration of data stored in the EMR storage 106 of the primary EMR server 101 with a plurality of third-party EMR servers 111. For example, the EMR interface application 103 may be referred to as an adapter that plugs into a computing device, such as the primary EMR server 101, to interface and connect with other servers 111 for sending and receiving data including EMR. In some embodiments, the EMR interface application 103 can be implemented using programmable or specialized hardware, such as a field-programmable gate array (FPGA) or an application-specific integrated circuit (ASIC). In some embodiments, the EMR interface application 103 can be implemented using a combination of hardware and software. In other embodiments, the EMR interface application 103 may be stored and executed on a combination of the client devices 115 and the primary EMR server 101, or by any one of the client devices 115 or primary EMR server 101.

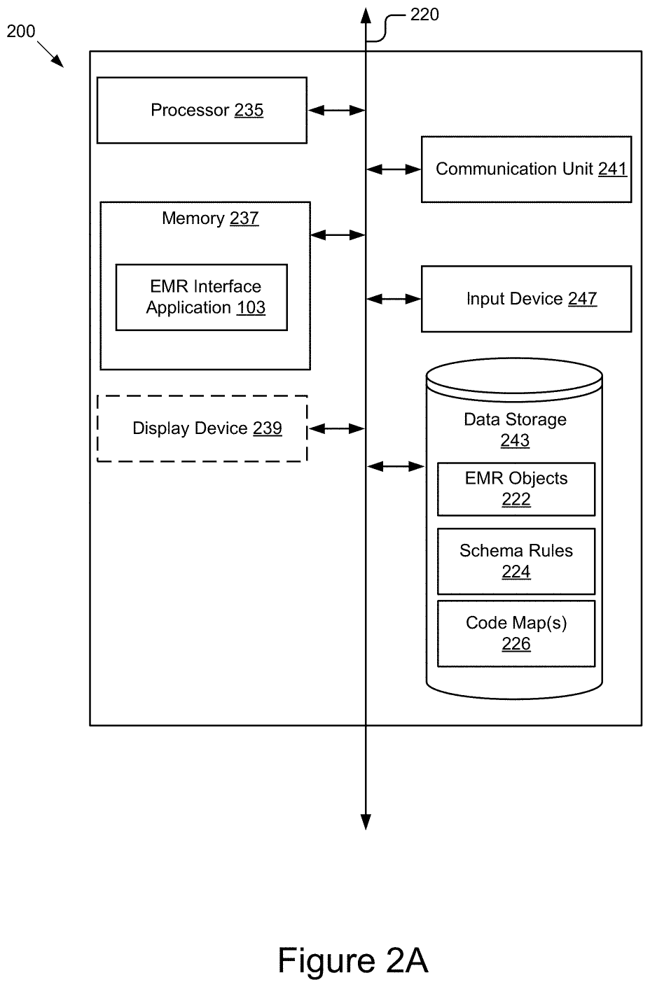

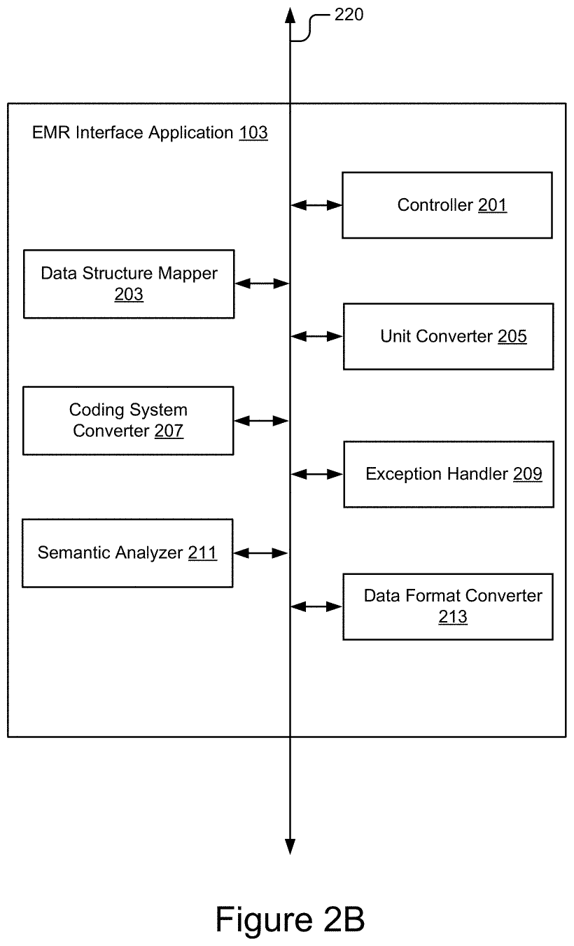

In some embodiments, the EMR interface application 103a may be a thin-client application with some functionality executed on the client device 115a and additional functionality executed on the primary EMR server 101 by the EMR interface application 103b. For example, the EMR interface application 103a on the client device 115 could include software and/or logic for resolving an exception and transmitting the resolution to the primary EMR server 101. In another example, the EMR interface application 103b on the primary EMR server 101 could include software and/or logic for receiving EMR data in a first data format, parsing elements of the EMR data, reorganizing into an intermediate data format of a standard schema, and converting the standard schema of the intermediate data format into a second data format. The operation of the EMR interface application 103 and the functions listed above are described below in more detail below with reference to FIGS. 2-6.

FIG. 2A is a block diagram illustrating one embodiment of a computing device 200 including an EMR interface application 103. The computing device 200 may also include a processor 235, a memory 237, an optional display device 239, a communication unit 241, an input device 247, and a data storage 243 according to some examples. The components of the computing device 200 are communicatively coupled by a bus 220. The bus 220 may represent one or more buses including an industry standard architecture (ISA) bus, a peripheral component interconnect (PCI) bus, a universal serial bus (USB), or some other bus known in the art to provide similar functionality. In some embodiments, the computing device 200 may be the client device 115, the primary EMR server 101, or a combination of the client device 115 and the primary EMR server 101. In such embodiments where the computing device 200 is the client device 115 or the primary EMR server 101, it should be understood that the client device 115, and the primary EMR server 101 may include other components described above but not shown in FIG. 2A.

The processor 235 may execute software instructions by performing various input/output, logical, and/or mathematical operations. The processor 235 may have various computing architectures to process data signals including, for example, a complex instruction set computer (CISC) architecture, a reduced instruction set computer (RISC) architecture, and/or an architecture implementing a combination of instruction sets. The processor 235 may be physical and/or virtual and may include a single processing unit or a plurality of processing units and/or cores. In some implementations, the processor 235 may be capable of generating and providing electronic display signals to a display device, supporting the display of images, capturing and transmitting images, performing complex tasks including various types of feature extraction and sampling, etc. In some implementations, the processor 235 may be coupled to the memory 237 via the bus 220 to access data and instructions therefrom and store data therein. The bus 220 may couple the processor 235 to the other components of the computing device 200 including, for example, the memory 237, the communication unit 241, the EMR interface application 103, and the data storage 243. It will be apparent to one skilled in the art that other processors, operating systems, sensors, displays, and physical configurations are possible.

The memory 237 may store and provide access to data for the other components of the computing device 200. The memory 237 may be included in a single computing device or distributed among a plurality of computing devices as discussed elsewhere herein. In some implementations, the memory 237 may store instructions and/or data that may be executed by the processor 235. The instructions and/or data may include code for performing the techniques described herein. For example, in one embodiment, the memory 237 may store the EMR interface application 103 executable by the processor 235. The memory 237 is also capable of storing other instructions and data, including, for example, an operating system, hardware drivers, other software applications, databases, etc. The memory 237 may be coupled to the bus 220 for communication with the processor 235 and the other components of the computing device 200.

The memory 237 may include one or more non-transitory computer-usable (e.g., readable, writeable) device, a static random access memory (SRAM) device, a dynamic random access memory (DRAM) device, an embedded memory device, a discrete memory device (e.g., a PROM, FPROM, ROM), a hard disk drive, an optical disk drive (CD, DVD, Blu-ray™, etc.) mediums, which can be any tangible apparatus or device that can contain, store, communicate, or transport instructions, data, computer programs, software, code, routines, etc., for processing by or in connection with the processor 235. In some implementations, the memory 237 may include one or more of volatile memory and non-volatile memory. It should be understood that the memory 237 may be a single device or may include multiple types of devices and configurations.

The display device 239 is a liquid crystal display (LCD), light emitting diode (LED) or any other similarly equipped display device, screen or monitor. The display device 239 represents any device equipped to display user interfaces, electronic images, and data as described herein. In different embodiments, the display is binary (only two different values for pixels), monochrome (multiple shades of one color), or allows multiple colors and shades. The display device 239 is coupled to the bus 220 for communication with the processor 235 and the other components of the computing device 200. It should be noted that the display device 239 is shown in FIG. 2 with dashed lines to indicate it is optional. For example, where the computing device 200 is the primary EMR server 101, the display device 239 may not be part of the system, where the computing device 200 is the client device 115, the display device 239 is included and is used to display user interfaces for performing the techniques described herein.

The input device 247 may include any device for inputting information into the computing device 200. In some implementations, the input device 247 may include one or more peripheral devices. For example, the input device 247 may include a keyboard (e.g., a QWERTY keyboard), a pointing device (e.g., a mouse or touchpad), a microphone, an image/video capture device (e.g., camera), etc. In some implementations, the input device 247 may include a touch-screen display capable of receiving input from the one or more fingers of the user. For instance, the functionality of the input device 247 and the display 239 may be integrated, and a user of the computing device 200 (e.g., client device 115) may interact with the computing device 200 by contacting a surface of the display device 239 using one or more fingers. In this example, the user can interact with an emulated (i.e., virtual or soft) keyboard displayed on the touch-screen display device 239 by using fingers to contact the display in the keyboard regions.

The communication unit 241 is hardware for receiving and transmitting data by linking the processor 235 to the network 105 and other processing systems. The communication unit 241 receives data such as requests from the client device 115 and transmits the requests to the components of the EMR interface application 103. The communication unit 241 is coupled to the bus 220. In one embodiment, the communication unit 241 may include a port for direct physical connection to the client device 115 or to another communication channel. For example, the communication unit 241 may include an RJ45 port or similar port for wired communication with the client device 115. In another embodiment, the communication unit 241 may include a wireless transceiver (not shown) for exchanging data with the client device 115 or any other communication channel using one or more wireless communication methods, such as IEEE 802.11, IEEE 802.16, Bluetooth® or another suitable wireless communication method.

In yet another embodiment, the communication unit 241 may include a cellular communications transceiver for sending and receiving data over a cellular communications network such as via short messaging service (SMS), multimedia messaging service (MMS), hypertext transfer protocol (HTTP), direct data connection, WAP, e-mail or another suitable type of electronic communication. In still another embodiment, the communication unit 241 may include a wired port and a wireless transceiver. The communication unit 241 also provides other conventional connections to the network 105 for distribution of files and/or media objects using standard network protocols such as TCP/IP, HTTP, HTTPS, and SMTP as will be understood to those skilled in the art.

The data storage 243 is a non-transitory memory that stores data for providing the functionality described herein. The data storage 243 may be coupled to the components of the EMR interface application 103 via the bus 220 to receive and provide access to data. In some embodiments, the data storage 243 may store data received from other elements of the system 100, including, for example, the client device 115, the medical device 113, and/or the EMR interface application 103, and may provide data access to these entities. The data storage 243 may store, among other data, EMR objects 222, schema rules 224, and code maps 226. The EMR objects 222 represents the electronic medical record of patients stored in a file format such as JavaScript Object Notation (JSON), Extensible Markup Language (XML), etc. The schema rules 224 represent a set of rules for parsing and mapping a structure or format of a file, for example, an XML document into another structure or format, for example, a valid JSON document. The schema rules 224 may include schema mappings that define how data is to be converted between the schemas of a source data format and a target data format. The schema rules 224 may include XML schemas, such as, Document Type Definition (DTD), XML Schema Definition (XSD), and Relax-NG. The code maps 226 represent one or more conceptual mappings from one type of medical coding classification system to another. In the illustrated embodiment, the data storage 243 is communicatively coupled to the bus 220. The data stored in the data storage 243 is described below in more detail.

The data storage 243 may be included in the computing device 200 or in another computing device and/or storage system distinct from but coupled to or accessible by the computing device 200. The data storage 243 can include one or more non-transitory computer-readable mediums for storing the data. In some embodiments, the data storage 243 may be incorporated with the memory 237 or may be distinct therefrom. The data storage 243 may be a dynamic random access memory (DRAM) device, a static random access memory (SRAM) device, flash memory, or some other memory devices. In some embodiments, the data storage 243 also may include a non-volatile memory or similar permanent storage device and media including a hard disk drive, a floppy disk drive, a CD-ROM device, a DVD-ROM device, a DVD-RAM device, a DVD-RW device, a flash memory device, or some other mass storage device for storing information on a more permanent basis. In some embodiments, the data storage 243 may include a database management system (DBMS) operable on the computing device 200. For example, the DBMS could include a structured query language (SQL) DBMS, a NoSQL DBMS, various combinations thereof, etc. In some instances, the DBMS may store data in multi-dimensional tables comprised of rows and columns, and manipulate, e.g., insert, query, update and/or delete, rows of data using programmatic operations. In some other instances, the DBMS may be a document-oriented database program (e.g., MongoDB™) that stores data using JSON-like documents.

Referring now to FIG. 2B, the EMR interface application 103 is shown in more detail. FIG. 2B is a block diagram illustrating one embodiment of the EMR interface application 103.

In some embodiments, the EMR interface application 103 may include a controller 201, a data structure mapper 203, a unit converter 205, a coding system converter 207, an exception handler 209, a semantic analyzer 211, and a data format converter 213. The components of the EMR interface application 103 are communicatively coupled via the bus 220. The components of the EMR interface application 103 may each include software and/or logic to provide their respective functionality. In some embodiments, the components of the EMR interface application 103 can each be implemented using programmable or specialized hardware including a field-programmable gate array (FPGA) or an application-specific integrated circuit (ASIC). In some embodiments, the components of the EMR interface application 103 can each be implemented using a combination of hardware and software executable by the processor 235. In some embodiments, the components of the EMR interface application 103 may each be stored in the memory 237 and be accessible and executable by the processor 235. In some embodiments, the components of the EMR interface application 103 may each be adapted for cooperation and communication with the processor 235, the memory 237, and other components of the EMR interface application 103 via the bus 220.

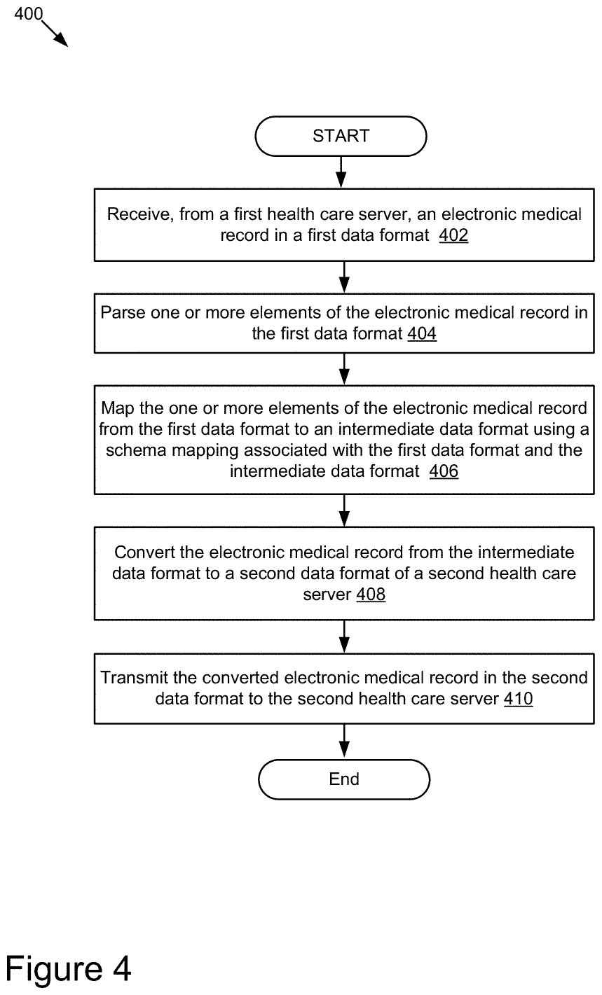

The controller 201 may include software and/or logic to control the operation of the other components of the EMR interface application 103. The controller 201 controls the other components of the EMR interface application 103 to perform the methods described below with reference to FIGS. 4-6. The controller 201 may also include software and/or logic to provide the functionality for handling communications between the EMR interface application 103 and other components of the computing device 200 as well as between the components of the EMR interface application 103.

In some embodiments, the controller 201 sends and receives data, via the communication unit 241, to and from one or more of the client device 115, the primary EMR server 101, and the third-party EMR severs 111. For example, the controller 201 receives, via the communication unit 241, a request to transfer a patient's EMR to another hospital from a client device 115 operated by a user and sends the request to the data structure mapper 203. In some embodiments, the controller 201 receives data from other components of the EMR interface application 103 and stores the data in the data storage 243. For example, the controller 201 receives data including an EMR object for a patient from the data format converter 213 and stores the data in the data storage 243. In other embodiments, the controller 201 retrieves data from the data storage 243 and sends the data to other components of the EMR interface application 103. For example, the controller 201 retrieves data including a code map 226 from the data storage 243 and sends the retrieved data to the coding system converter 207.

In some embodiments, the communications between the EMR interface application 103 and other components of the computing device 200 as well as between the components of the EMR interface application 103 can occur autonomously and independent of the controller 201.

The data structure mapper 203 may include software and/or logic to provide the functionality for transforming or mapping a structure of a data object from one data format to another data format. In some embodiments, the data structure mapper 203 may retrieve an EMR object in a source data format, such as JSON format from the data storage 243. The EMR object may be retrieved in response to a request for transferring the EMR object to an external health care server of a different health care organization. In some embodiments, the data structure mapper 203 retrieves schema rules 224 from the data storage 243 for parsing the data object. The schema rules 224 may include one or more schema mappings and XML schemas. A schema mapping may be a specification defining a relationship or correspondence between the elements of the schemas of source data format (e.g., JSON) and intermediate data format (e.g., XML). The XML schema specifies a structure and content of the XML data format for validating an XML file or object. The data structure mapper 203 parses a structure of the data object and identifies one or more elements in the data object. The data structure mapper 203 maps the parsed elements in the data object from the source data format to an intermediate data format using the schema mapping. For example, the data structure mapper 203 reorganizes the parsed elements of the EMR object from JSON data format into the standard XML data format. In some embodiments, the data structure mapper 203 may access XML schema definition (XSD) in the XML schemas and check the mapped data object in the intermediate XML data format against XSD to determine if it is valid. If it is determined valid, the data structure mapper 203 sends the mapped data object to the data format converter 213.

In some embodiments, the data structure mapper 203 may receive a data object associated with an external health care sever of a different health care organization. The data object may be an incoming EMR object formatted in a destination data format (e.g., HL7 v 2.x) of the external health care server and converted to an intermediate data format (e.g., XML file) by the data format converter 213 described in detail below. The data structure mapper 203 may parse the received data object and map the parsed elements of the data object from intermediate data format to the source data format using the schema mapping. For example, the data structure mapper 203 reorganizes the parsed elements of the EMR object from standard XML data format into the JSON data format. The data structure mapper 203 stores the EMR data object mapped into the source data format in the EMR objects 222 of the data storage 243.

In some embodiments, the data structure mapper 203 may receive unit conversion and medical coding classification system conversion for one or more elements in the data object from the unit converter 205 and the coding system converter 207 described in more detail below. The data structure mapper 203 updates the elements of the data object with the conversions accordingly.

The unit converter 205 may include software and/or logic to provide the functionality for converting a unit of measurement of one or more elements in the data object from one system of measurement to another. For example, the systems of measurement may include the metric system, the imperial system, and the United States customary units. The unit converter 205 determines a first system of measurement used for representing the one or more elements in the data object. For example, the unit converter 205 may determine that the one or more elements in a data structure of the EMR object record the physical characteristics of a patient in the metric system. The physical characteristics may include a weight of a patient recorded in kilograms, a height of the patient recorded in meters, a temperature reading of the patient recorded in degree Celsius, etc. The unit converter 205 determines the requirements including a system of measurement units preferred by a destination health care server (e.g., third-party server 111) to which the data object (e.g., EMR object of the patient) is to be sent. The unit converter 205 determines a second system of measurement to represent the elements based on the requirements. For example, the unit converter 205 may determine that the requirements indicate the second system of measurement of the destination health care server to be United States customary units. The unit converter 205 converts the first system of measurement for the one or more elements in the data object to the second system of measurement. For example, the unit converter 205 converts the weight element in the EMR object represented in kilograms to pounds, the height element in the EMR object represented in meters to feet and inches, the temperature element in the EMR object represented in degree Celsius to degree Fahrenheit, and so on. In some embodiments, the unit converter 205 sends the one or more elements that have been converted to the new system of measurement to the data structure mapper 203.

The coding system converter 207 may include software and/or logic to provide the functionality for converting a medical coding classification system encoding one or more elements in the data object from one classification system to another. A medical coding classification system may encode the clinical terms of medical diagnoses, descriptions of symptoms, procedures of medical interventions, and other medical-related terms into alphanumeric medical code numbers enabling a consistent way of capturing, sharing, and aggregating health data across health information systems. For example, the medical coding classification system may include Systematized Nomenclature of Medicine-Clinical Terms (SNOMED-CT) and International Classification of Disease (ICD). ICD may further include different versions, such as ICD-9, ICD-10, ICD-11, etc. The coding system converter 207 determines a first medical coding classification system used to encode the one or more elements in the data object. For example, the coding system converter 207 may determine that a source health care server (e.g., primary EMR server 101) encodes one or more clinical elements in a data structure of the EMR object using SNOMED-CT classification system. The coding system converter 207 determines a second medical coding classification system used by a destination health care server to which the data object (e.g., EMR object of the patient) is to be sent. For example, the coding system converter 207 may determine that the destination health care server uses ICD-10 to encode clinical elements of the EMR object of a patient. The coding system converter 207 retrieves a code map 226 from the data storage 243 based on the first and the second medical coding classification system. For example, the code map may provide a mapping of codes from SNOMED-CT data model to corresponding codes in ICD-10 data model. The coding system converter 207 converts the medical-related codes of the one or more elements in the data object from the first medical coding classification system to the second medical coding classification system using the code map. In some embodiments, the unit converter 205 sends the one or more elements that have been converted to the new medical coding classification system to the data structure mapper 203.

The exception handler 209 may include software and/or logic to provide the functionality for handling exceptions that may arise during one or more of the data transformation, coding space conversion, and the unit conversion of the one or more elements of the data object.

In some embodiments, the data format mapper 203, the unit converter 205, and/or the coding system converter 207 may detect an inconsistency in performing one or more of the data transformation, coding space conversion, and the unit conversion of the one or more elements of the data object. For example, the code maps for mapping the different medical coding classification systems may not be precise or available. Alternatively, there may be multiple code maps available for converting from one medical coding classification system to another. In another example, the schema mapping may not be complete for performing a data transformation on the data object. The components 203, 205, and/or 207 of the EMR interface application 103 may send the detected inconsistency to the exception handler 209 for providing a resolution to the detected inconsistency in performing one or more of the data transformation, coding space conversion, and the unit conversion of the one or more elements of the data object. In some embodiments, the exception handler 209 generates an exception event based on the detected inconsistency. The exception handler 209 uses a machine learning system to process the exception event. For example, the machine learning system may be a dictionary learning system. In some embodiments, the exception handler 209 identifies a number of exception events as they get generated and determines how those exception events were handled and addressed by a user in the past. The exception handler 209 continuously curates an input data set using the exception events that occurred in the past and the resolutions provided by the user to those exceptions. The exception handler 209 uses an algorithm to continuously learn or train a dictionary to fit the input data set. For example, the algorithm may include method of optimal directions (MOD), k-singular value decomposition (K-SVD), stochastic gradient descent, lagrange dual method, and least absolute shrinkage and selection operator (LASSO), etc.

In some embodiments, the exception handler 209 uses a learned dictionary to automatically process the exception event, resolve the detected inconsistency in performing one or more of the data transformation, coding space conversion, and the unit conversion of the one or more elements of the data object, and send a resolution to the inconsistency in terms of the converted or transformed elements of the data object to the components 203, 205, and/or 207. In some embodiments, the exception handler 209 generates a notification of a resolution to the inconsistency that was generated using the dictionary and sends the notification to a user after the processing of the exception event. If the user disagrees with the resolution after the fact and provides his or her input overriding the resolution to the exception event from the machine learning system, the exception handler 209 updates the conversion or transformation of the elements of the data object based on the user input. The exception handler 209 also updates the input data set with the user input providing a resolution to the exception event.

In some embodiments, the exception handler 209 determines a context of the exception event. For example, the context may indicate that the exception event is triggered when a data object is subject to data transformation, coding space conversion, and/or unit conversion for transmitting the data object to a particular EMR server 111 or receiving the data object from a particular EMR server 111. Exception events associated with one target EMR server 111 may be different compared to the exception events associated with another target EMR server 111. The exception handler 209 uses a dictionary that matches the context of the exception event for processing the exception event. In other words, if the context in which the dictionary was learned by the exception handler 209 matches the context of the exception event, then the dictionary is used for processing the exception event. In some embodiments, the exception handler 209 stores one or more contextual dictionaries that are learned on one or more input data sets in the data storage 243.

In some embodiments, the exception handler 209 may not be able to process the exception event using the learned dictionary. In such instances, the exception handler 209 sends a notification of the exception event to a client device 115 of a user and requests the user to provide a resolution. For example, the exception event may request the user to choose a correct mapping between a SNOMED-CT code and an ICD-10 code in the instance that the code map includes one-to-many relationships or a many-to-one relationships between the clinical codes of SNOMED-CT and ICD-10. In another example, the exception event may be an issue in mapping the names and values in JSON and XML during the transformation of the data object from JSON to XML data format. The user may handle and provide a resolution to the exception event in real time. For example, the user may be an administrator of an EMR application in the primary EMR server 101. In another example, the user may be an authorized personnel within a health care organization associated with the primary EMR server 101. The exception handler 209 receives the resolution to the exception event from the user and sends the converted or transformed elements of the data object to the components 203, 205, and/or 207 of the EMR interface application 103.

The semantic analyzer 211 may include software and/or logic to provide the functionality for performing semantic analysis. In some embodiments, the semantic analyzer 211 may perform semantic analysis on the transformed data object from the data structure mapper 203. The semantic analysis may include type checking to determine whether the data types are converted in a manner that is consistent with their definition. For example, the data types are checked to ensure that they are compatible data types and used with operations that are defined for them.

The data format converter 213 may include software and/or logic to provide the functionality for converting the data object received in the intermediate data format to a destination data format and vice versa. In some embodiments, the data format converter 213 may be an off-the-shelf converter. An example of the off-the-shelf converter may be HL7 Application Programming Interface (HAPI). HAPI may include a library that provides pre-built support to enable message construction, parsing, validation, transmission and reception of a broad set of HL7 2.x standards and their message types. In some embodiments, the data format converter 213 receives the data object in the intermediate data format from the data format mapper 203 for transmitting the data object to a target health care server 111. For example, the data object may be an EMR object in standard XML data format. The data format converter 213 converts the intermediate data format of the data object to a destination data format used by the target health care server 111. For example, the destination data format may include one of several versions of HL7 message formatting standard, such as HL7 version 2.1, version 2.2, version 2.3, version 2.3.1, version 2.4, etc. Furthermore, a first destination health care server 111 using Epic® EMR application may implement HL7 version 2.3.1 in a manner that is different from a second destination health care server 111 using Centricity® EMR application. Although only one data format converter 213 is depicted in FIG. 2B, there may be separate instances of data format converters 213 connected to the bus 220. Each instance of the data format converter 213 connects to a different destination health care server 111 and converts the data object in the intermediate data format to one of HL7 versions used by the destination health care sever 111. The data format converter 213 validates the data object in destination data format HL7 messages for syntax and semantics before the converted data object is sent to the destination health care server 111. In some embodiments, the data format converter 213 receives the incoming data objects in HL7 message format from the destination health care server 111, converts them to intermediate XML data format, and sends the data objects in XML data format to the data format mapper 203 for further processing.

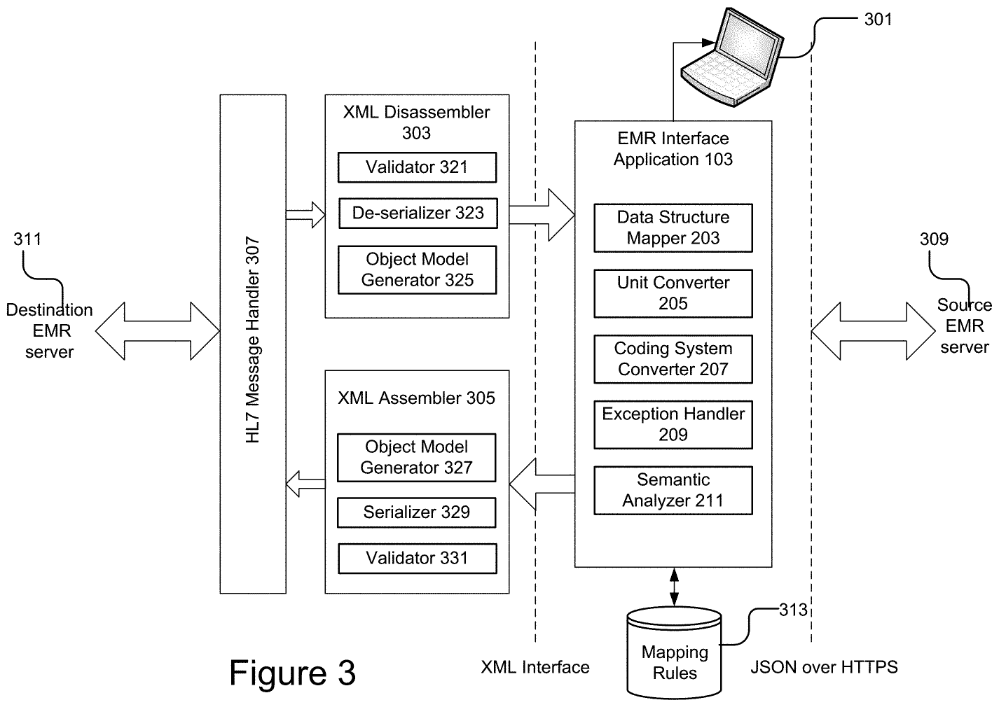

FIG. 3 is a block diagram illustrating another embodiment of the EMR interface application 103. The EMR interface application 103 receives a data object, such as an EMR object, from source EMR server 309 for transmitting to a destination EMR server 311. In some embodiments, the EMR interface application 103 includes the data structure mapper 203, the unit converter 205, the coding system converter 207, the exception handler 209, and the semantic analyzer 211. The data structure mapper 203 accesses mapping rules 313 and maps a structure of the EMR object from a source data format, such as JSON, to an intermediate data format, such as XML and vice versa. The unit converter 205 converts a unit of measurement for one or more elements of the EMR object from a first system of measurement to a second system of measurement applicable to the destination EMR server 311. The coding system converter 207 converts a medical coding classification system encoding one or more clinical elements of the EMR object from a first medical coding classification system, such as SNOMED-CT, to a second medical coding classification system, such as ICD-10. The exception handler 209 generates an exception event based on an inconsistency detected in one or more of data format conversion, unit conversion, and coding space conversion. The exception handler 209 sends a notification of the exception event to a client device 301 of a user. The user may submit or approve a modification that reconciles and fixes the issue associated with the inconsistency. The semantic analyzer 211 performs semantic analysis on the mapped data object.

In some embodiments, the EMR interface application 103 makes use of the XML assembler 305 and XML disassembler 303 for mapping the data object from the source data format to the intermediate data format and vice versa. The XML assembler 305 includes an object model generator 327, a serializer 329, and a validator 331. The object model generator 327 generates an object model from the mapped data object in the XML data format that is consistent for the destination EMR server 311. The serializer 329 serializes the object to the XML file. Serialization converts the object into a form that may be readily transported. The validator 331 validates the XML file before it is sent to the HL7 Message Handler 307. This prevents the sending of mal-formatted XML file to the HL7 Message Handler 307. The XML assembler 305 sends the XML file to the HL7 message handler 307. The HL7 message handler 307 is the data format converter 213 from FIG. 2B. An example of the HL7 Message Handler 307 may be HL7 Application Programming Interface (HAPI). The HL7 message handler 307 converts the XML file to HL7 destination format which is then sent to the destination EMR server 311.

In some embodiments, the HL7 message handler 307 receives an incoming data object in the HL7 message format from the destination EMR server 311 and converts the data object in the HL7 message format to an XML file. The HL7 message handler 307 sends the XML file to the XML disassembler 303. The XML disassembler 303 includes a validator 321, de-serializer 323, and object model generator 325. The validator 321 validates the XML file. The de-serializer 323 deserializes the XML file back to an object. Deserialization converts converts the byte of data, such as the XML file to object type. The object model generator 325 generates the object model for JSON. The XML disassembler 303 sends the JSON object to the EMR interface application 103. The EMR interface application 103 converts the unit measurement system for the data object to one that is used by the source EMR server 309 and converts the coding space for the data object to one that is used by the source EMR server 309. The EMR interface application 103 sends the JSON data object to the source EMR server 309 for storage.

FIG. 4 is a flow diagram illustrating one embodiment of an example method 400 for providing an interface to facilitate interoperability between health information systems. At 402, the data format mapper 203 receives an electronic medical record in a first data format from a first health care server. At 404, the data format mapper 203 parses one or more elements of the electronic medical record in the first data format. At 406, the data format mapper 203 map the one or more elements of the electronic medical record from the first data format to an intermediate data format using a schema mapping associated with the first data format and the intermediate data format. At 408, the data format converter 213 convert the electronic medical record from the intermediate data format to a second data format of a second health care server. At 410, the data format converter 213 transmit the converted electronic medical record in the second data format to the second health care server.

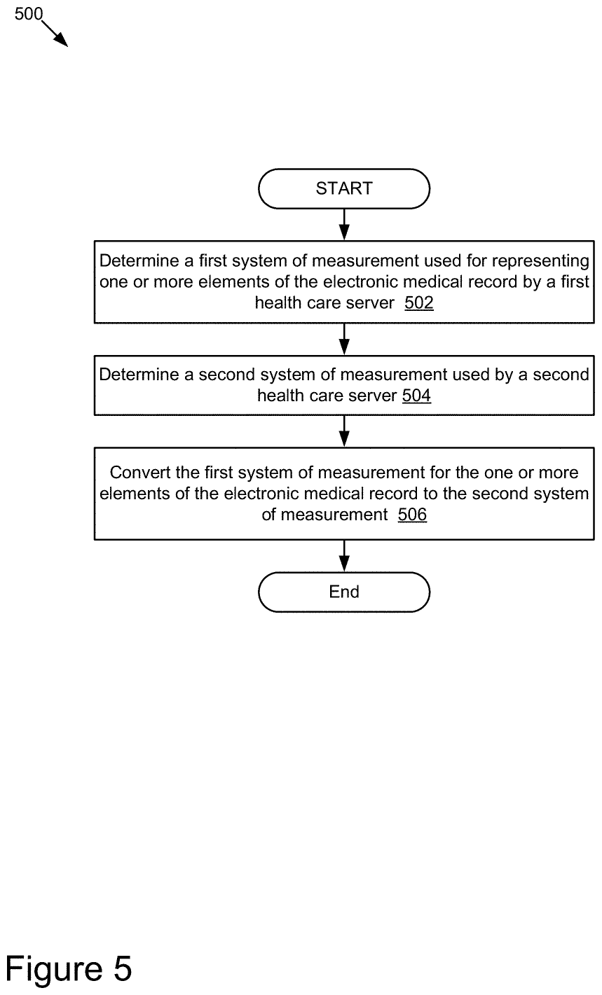

FIG. 5 is a flow diagram illustrating one embodiment of an example method 500 for converting a system of measurement of the elements of a data object. At 502, the unit converter 205 determines a first system of measurement used for representing one or more elements of the electronic medical record by a first health care server. At 504, the unit converter 205 determines a second system of measurement used by a second health care server. At 506, the unit converter 205 converts the first system of measurement for the one or more elements of the electronic medical record to the second system of measurement.

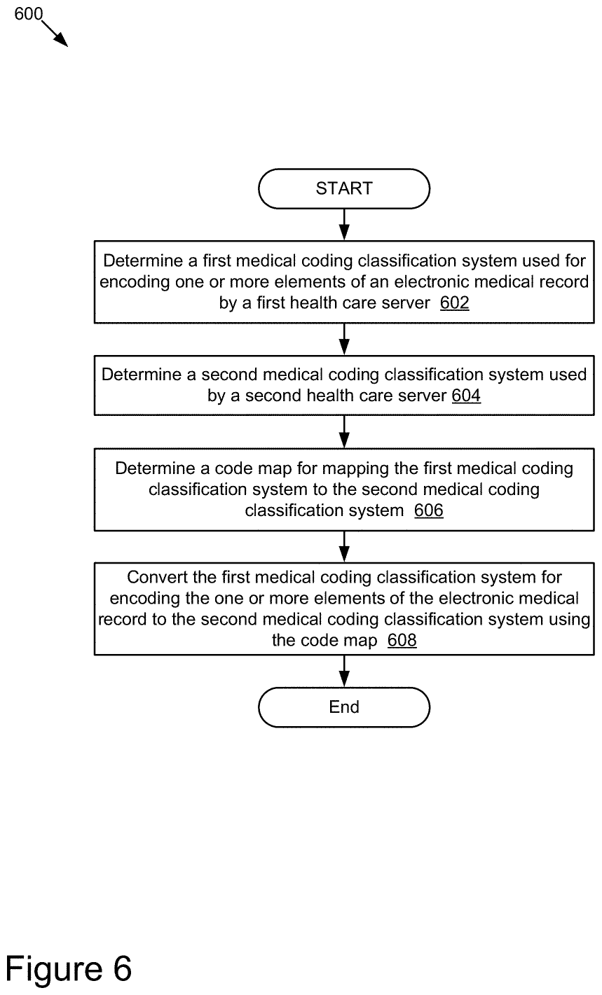

FIG. 6 is a flow diagram illustrating one embodiment of an example method 600 for converting a medical coding classification system of the elements of a data object. At 602, the coding system converter 207 determines a first medical coding classification system used for encoding one or more elements of an electronic medical record by a first health care server. At 604, the coding system converter 207 determines a second medical coding classification system used by a second health care server. At 606, the coding system converter 207 determines a code map for mapping the first medical coding classification system to the second medical coding classification system. At 608, the coding system converter 207 converts the first medical coding classification system for encoding the one or more elements of the electronic medical record to the second medical coding classification system using the code map.

A system and method for providing an interface to facilitate interoperability between health information systems has been described. In the above description, for purposes of explanation, numerous specific details are set forth in order to provide a thorough understanding of the techniques introduced above. It will be apparent, however, to one skilled in the art that the techniques can be practiced without these specific details. In other instances, structures and devices are shown in block diagram form in order to avoid obscuring the description and for ease of understanding. For example, the techniques are described in one embodiment above primarily with reference to software and particular hardware. However, the present invention applies to any type of computing system that can receive data and commands, and present information as part of any peripheral devices providing services.

Reference in the specification to “one embodiment” or “an embodiment” means that a particular feature, structure, or characteristic described in connection with the embodiment is included in at least one embodiment. The appearances of the phrase “in one embodiment” in various places in the specification are not necessarily all referring to the same embodiment.

Some portions of the detailed descriptions described above are presented in terms of algorithms and symbolic representations of operations on data bits within a computer memory. These algorithmic descriptions and representations are, in some circumstances, used by those skilled in the data processing arts to convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing”, “computing”, “calculating”, “determining”, “displaying”, or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques also relate to an apparatus for performing the operations herein. This apparatus may be specially constructed for the required purposes, or it may comprise a general-purpose computer selectively activated or reconfigured by a computer program stored in the computer. Such a computer program may be stored in a non-transitory computer readable storage medium, such as, but is not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

Some embodiments can take the form of an entirely hardware embodiment, an entirely software embodiment or an embodiment containing both hardware and software elements. One embodiment is implemented in software, which includes but is not limited to firmware, resident software, microcode, etc.

Furthermore, some embodiments can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

A data processing system suitable for storing and/or executing program code can include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description above. In addition, the techniques are not described with reference to any particular programming language. It will be appreciated that a variety of programming languages may be used to implement the teachings of the various embodiments as described herein.

The foregoing description of the embodiments has been presented for the purposes of illustration and description. It is not intended to be exhaustive or to limit the specification to the precise form disclosed. Many modifications and variations are possible in light of the above teaching. It is intended that the scope of the embodiments be limited not by this detailed description, but rather by the claims of this application. As will be understood by those familiar with the art, the examples may be embodied in other specific forms without departing from the spirit or essential characteristics thereof. Likewise, the particular naming and division of the modules, routines, features, attributes, methodologies and other aspects are not mandatory or significant, and the mechanisms that implement the description or its features may have different names, divisions and/or formats. Furthermore, as will be apparent to one of ordinary skill in the relevant art, the modules, routines, features, attributes, methodologies and other aspects of the specification can be implemented as software, hardware, firmware or any combination of the three. Also, wherever a component, an example of which is a module, of the specification is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming. Additionally, the specification is in no way limited to embodiment in any specific programming language, or for any specific operating system or environment. Accordingly, the disclosure is intended to be illustrative, but not limiting, of the scope of the specification, which is set forth in the following claims.

What is claimed is:

1. A method comprising:
receiving, from a first health care server, an electronic medical record in a first data format;
parsing one or more elements of the electronic medical record in the first data format;
mapping the one or more elements of the electronic medical record from the first data format to an intermediate data format using a schema mapping associated with the first data format and the intermediate data format;
converting the electronic medical record from the intermediate data format to a second data format of a second health care server; and
transmitting the converted electronic medical record in the second data format to the second health care server.

2. The method of claim 1, wherein mapping the one or more elements of the electronic medical record further comprises:
determining a first system of measurement used for representing the one or more elements of the electronic medical record by the first health care server;
determining a second system of measurement used by the second health care server; and
converting the first system of measurement for the one or more elements of the electronic medical record to the second system of measurement.

3. The method of claim 1, wherein mapping the one or more elements of the electronic medical record further comprises:
determining a first medical coding classification system used for encoding the one or more elements of the electronic medical record by the first health care server;
determining a second medical coding classification system used by the second health care provider;
determining a code map for mapping the first medical coding classification system to the second medical coding classification system; and
converting the first medical coding classification system for encoding the one or more elements of the electronic medical record to the second medical coding classification system using the code map.

4. The method of claim 1, further comprising:
determining an inconsistency in mapping the one or more elements of the electronic medical record;
generating an exception event based on the inconsistency;
determining a learned dictionary;
processing the exception event using the learned dictionary; and
resolving the inconsistency in mapping the one or more elements of the electronic medical record based on processing the exception event using the learned dictionary.

5. The method of claim 1, wherein the first data format is JavaScript Object Notation (JSON), the intermediate data format is extensible markup language (XML), and the second data format is health level 7 message format.

6. The method of claim 2, wherein one or more of the first system of measurement and the second system of measurement is a system of measurement from a group of metric system, imperial system, and United States customary units.

7. The method of claim 3, wherein one or more of the first medical coding classification system and the second medical coding classification system is a medical coding classification system from a group of Systematized Nomenclature of Medicine (SNOMED) and International Classification of Disease (ICD).

8. A system comprising:
one or more processors; and
a memory, the memory storing instructions, which when executed cause the one or more processors to:
receive, from a first health care server, an electronic medical record in a first data format;
parse one or more elements of the electronic medical record in the first data format;
map the one or more elements of the electronic medical record from the first data format to an intermediate data format using a schema mapping associated with the first data format and the intermediate data format;
convert the electronic medical record from the intermediate data format to a second data format of a second health care server; and
transmit the converted electronic medical record in the second data format to the second health care server.

9. The system of claim 8, wherein to map the one or more elements of the electronic medical record, the instructions further cause the one or more processors to:
determine a first system of measurement used for representing the one or more elements of the electronic medical record by the first health care server;
determine a second system of measurement used by the second health care server; and
convert the first system of measurement for the one or more elements of the electronic medical record to the second system of measurement.

10. The system of claim 8, wherein to map the one or more elements of the electronic medical record, the instructions further cause the one or more processors to:
determine a first medical coding classification system used for encoding the one or more elements of the electronic medical record by the first health care server;
determine a second medical coding classification system used by the second health care provider;
determine a code map for mapping the first medical coding classification system to the second medical coding classification system; and
convert the first medical coding classification system for encoding the one or more elements of the electronic medical record to the second medical coding classification system using the code map.

11. The system of claim 8, wherein the instructions further cause the one or more processors to:
determine an inconsistency in mapping the one or more elements of the electronic medical record;
generate an exception event based on the inconsistency;
determine a learned dictionary;
process the exception event using the learned dictionary; and
resolve the inconsistency in mapping the one or more elements of the electronic medical record based on processing the exception event using the learned dictionary.

12. The system of claim 8, wherein the first data format is JavaScript Object Notation (JSON), the intermediate data format is extensible markup language (XML), and the second data format is health level 7 message format.

13. The system of claim 9, wherein one or more of the first system of measurement and the second system of measurement is a system of measurement from a group of metric system, imperial system, and United States customary units.

14. The system of claim 10, wherein one or more of the first medical coding classification system and the second medical coding classification system is a medical coding classification system from a group of Systematized Nomenclature of Medicine (SNOMED) and International Classification of Disease (ICD).

15. A computer program product comprising a non-transitory computer readable medium storing a computer readable program, wherein the computer readable program when executed on a computer causes the computer to:
receive, from a first health care server, an electronic medical record in a first data format;
parse one or more elements of the electronic medical record in the first data format;
map the one or more elements of the electronic medical record from the first data format to an intermediate data format using a schema mapping associated with the first data format and the intermediate data format;
convert the electronic medical record from the intermediate data format to a second data format of a second health care server; and
transmit the converted electronic medical record in the second data format to the second health care server.

16. The computer program product of claim 15, wherein to map the one or more elements of the electronic medical record, the computer readable program when executed on the computer further causes the computer to:
determine a first system of measurement used for representing the one or more elements of the electronic medical record by the first health care server;
determine a second system of measurement used by the second health care server; and
convert the first system of measurement for the one or more elements of the electronic medical record to the second system of measurement.

17. The computer program product of claim 15, wherein to map the one or more elements of the electronic medical record, the computer readable program when executed on the computer further causes the computer to:
determine a first medical coding classification system used for encoding the one or more elements of the electronic medical record by the first health care server;
determine a second medical coding classification system used by the second health care provider;
determine a code map for mapping the first medical coding classification system to the second medical coding classification system; and
convert the first medical coding classification system for encoding the one or more elements of the electronic medical record to the second medical coding classification system using the code map.

18. The computer program product of claim 15, wherein the computer readable program when executed on the computer further causes the computer to:
determine an inconsistency in mapping the one or more elements of the electronic medical record;
generate an exception event based on the inconsistency;
determine a learned dictionary;
process the exception event using the learned dictionary; and
resolve the inconsistency in mapping the one or more elements of the electronic medical record based on.

19. The computer program product of claim 15, wherein the first data format is JavaScript Object Notation (JSON), the intermediate data format is extensible markup language (XML), and the second data format is health level 7 message format.

20. The computer program product of claim 16, wherein one or more of the first system of measurement and the second system of measurement is a system of measurement from a group of metric system, imperial system, and United States customary units.
